(19) United States
(12) Patent Application Publication
(10) Pub. No.: US 2025/0346884 A1
(43) Pub. Date: Nov. 13, 2025

(54) METHOD OF DNA FRAGMENT SIZE SELECTION

(71) Applicants: QIAGEN GMBH, Hilden (DE); QIAGEN SCIENCES, LLC, Germantown, MD (US)

(72) Inventors: Thorsten SINGER, Solingen (DE); Siegfried HAUCH, Erkrath (DE); Nadja RATTE, Duesseldorf (DE); Nathan BLEWETT, Frederick, MD (US)

(21) Appl. No.: 18/863,515

(22) PCT Filed: May 11, 2023
(86) PCT No.: PCT/EP2023/062680

§ 371 Date: Nov. 6, 2024

(30) Foreign Application Priority Data
May 11, 2022 (EP) 22172791.0

(51) Int. Cl. C12N 15/10

(52) U.S. Cl. CPC C12N 15/1013

(57) ABSTRACT

The present invention concerns a method for isolating DNA molecules having a size above a certain cut-off value from a DNA-containing sample. The method comprises a) contacting the sample with an aqueous composition comprising beads with a negatively charged surface, a molecular crowding agent, a dissolved salt comprising at least one divalent metal cation, and optionally a buffer for a time sufficient to bind DNA to the surface of the beads; b) separating the beads with bound DNA from the remaining composition; c) optionally washing the beads; and d) optionally eluting the bound DNA from the beads; wherein the ratio of the concentration of salt to the concentration of beads in the aqueous composition in step a) is at a value where an increase in the ratio leads to an increase in said cut-off value.

### FIELD OF THE INVENTION

The present invention concerns a method for isolating DNA molecules having a size above a certain cut-off value from a DNA-containing sample. The method comprises the use of divalent metal cations and beads with a negatively charged surface, which allows for different cut-off values depending on the concentration of the divalent cations and/or the beads.

### BACKGROUND OF THE INVENTION

Illumina's Next generation sequencing workflow requires DNA with a size distribution of about 300-600 bp for optimal results. Especially smaller fragments will be sequenced without exploiting the whole possible read length and therefore wasting sequencing capacity. Furthermore, during the library preparation process, adapters are ligated to the end of the DNA but tend to dimerize resulting in a large fraction of 130 bp DNA fragments occupying sequencing capacity on the flow cell. Therefore, a size selective purification step (“size selection”) is required to get rid of these sequencing adapter dimers and smaller DNA fragments. This is typically achieved with a size selective polyethylene (PEG)-based precipitation or PEG-based size selective binding to solid-phase reversible immobilization (SPRI) (carboxylated) magnetic beads, e.g. AMPure XP Beads or QIAseq Beads as described in U.S. Pat. No. 6,534,262 B1. A size selection is achieved when using said beads by adding different amounts of carboxylated beads in suspension containing PEG8000 and NaCl in the molar range. An increase in the concentration of PEG8000/NaCl generally results in the binding of smaller and smaller fragments.

Rodrigue et al. (PLoSONE, 5, 2010, e11840) describe a method of isolating DNA below a certain cut-off value, based on the well-known sodium-based chemistry. WO 2013/045434 and WO 2014/122288 disclose a method of isolating DNA above a certain cut-off value based on the pH value of the binding mixture.

Stortchevoi et al. (Journal of Biomolecular Techniques, 31, 2020, 7-10) describe a PEG-based size selection in the kbp-range with carboxylated beads by exchanging the Na⁺ of the above described systems with different ions, such as Mg²⁺, Ca²⁺, and Li⁺. This paper confirms the general trend that an increase in the concentration of PEG8000/salt generally results in the binding of smaller and smaller fragments. It furthermore confirms that although generally a tuneable cut-off can be achieved using divalent cations, such as Mg²⁺ and Ca²⁺, small changes in cation concentration can lead to large changes in cut-off value. This makes this system highly sensible for even small pipetting errors resulting in a change in cut-off and reduced sequencing results.

The Beckmann-Coulter SPRIselect User Guide (https://research.fredhutch.org/content/dam/stripe/hahn/methods/mol_biol/SPRIselect%20User%2 0Guide.pdf) mentions the possible use of MgCl₂ as a salt (in addition to NaCl) and confirms the trend observed by Stortchevoi et al.

US 2018/0291365 A1 describes a size selection system using divalent cations, and high PEG concentrations in combination with unmodified silica beads at a pH between 8 and 10 and U.S. Pat. No. 10,745,686 B2 describes a size selective DNA isolation system based on chaotropic binding and variable pH for tunable cut-off.

However, these technologies are only able to remove fragments in the range of a few hundred base pairs which is usually sufficient for standard short-read sequencing (e.g. based on the Illumina technology). But a further dilution of the PEG solution to achieve larger cut-off results in a complete loss of binding before a cut-off in the kbp range is reached.

Different to the Illumina's short-read technology the so called third generation sequencing technologies—e.g. from Pacific Biosciences (PacBio) or Oxford Nanopore Technologies (ONT)—allow very long read lengths up to the mega-base range (ONT). Analogous to Illumina sequencing, the DNA also has to be in a certain size range to get best results. But the fragment size of the to be sequenced DNA is much higher in the range of tens of kbp and, therefore, a size selection to get rid of the smaller fragments here has indeed to remove all DNA fragments in the range of a few kilobases instead of only a few hundred bases.

Consequently, the technologies discussed above generally are suitable for standard short-read (Illumina) sequencing but do not fulfill the requirements of long read sequencing technologies.

To address this need, further new technologies were developed in the last years that were intended to achieve this size selection requirement in different ways.

WO 2019/006321A1 describes a technology which, e.g. in example 5, is based on the use of a solution of polyvinylpyrollidone 360.000 (PVP) and NaCl and is supposed to be realized in the Circulomics' Short Read Eliminator (SRE) which is commercially available with different cut-offs (SRE, SRE XL, SRE XS). Analogous to the SPRI bead formulation described above, a tunable size selection in a higher size range can be achieved. The principle is analogous to the PEG system described above: the higher the PVP/NaCl concentrations are the smaller the bound/precipitated fragments. The system described in WO 2019/006321 is furthermore based on the use of a nanomembrane rather than beads.

However, said SRE technology is highly dependent on the concentration of the input DNA and seems to not represent a real size selection technology with a defined cut-off. Instead, it rather uses a weak binding to lose all fragments with reduced representation in the sample. This means that short fragments <25kbp are progressively depleted and the overall recovery efficiency is dependent on the input DNA concentration. Besides that, because the procedure is based on size dependent precipitation, it cannot be integrated in a fully automated library preparation. Furthermore, the system is not generally tunable. The three versions of the SRE have their own progressively depleted range.

In consequence, currently there is no PEG-based technology available which fulfills the requirements and needs of size selection when using the new long-read sequencing technologies.

The inventors of the present invention have surprisingly found that if the divalent cation concentration (relative to the number of beads) in a system corresponding to the technology of Stortchevoi as described above is increased beyond the concentration where even the smallest fragments are bound, the opposite effect is observed, i.e. the cut-off value increases with a further increasing concentration of the divalent cation. Furthermore, at these higher concentrations the method is less sensitive to pipetting errors because small changes in concentration only lead to small changes in the cut-off value. It has furthermore been found that cut-off values of several kbp's can be achieved using the method of the invention and that these values can be adjusted by adjusting several different parameters of the method, making the method very versatile. The method of the invention thus allows the optimal preparation of DNA samples for third generation sequencing technologies. It has furthermore been found that non-bound fragments can be isolated in standard DNA purification systems, such as the QIAquick PCR Purification (QIAGEN, Hilden, Germany) as well and be used for e.g. additional parallel short read sequencing analysis.

### SUMMARY OF THE INVENTION

Accordingly, one aspect of the invention concerns a method for isolating DNA molecules having a size above a certain cut-off value from a DNA-containing sample, comprising
a) contacting the sample with an aqueous composition comprising beads with a negatively charged surface, a molecular crowding agent, a dissolved salt comprising at least one divalent metal cation, and optionally a buffer for a time sufficient to bind DNA to the surface of the beads;
b) separating the beads with bound DNA from the remaining composition;
c) optionally washing the beads; and
d) optionally eluting the bound DNA from the beads;
   wherein the ratio of the concentration of salt to the concentration of beads in the aqueous composition in step a) is at a value where an increase in the ratio leads to an increase in said cut-off value.

Thus, the invention utilizes the unexpected fact that there is a stationary point, in this case a minimum, in the cut-off value as a function of the ratio of the divalent metal cation concentration to the concentration of the beads, and that the cut-off value can be adjusted upwards beyond the stationary point. As will be set out infra, the location of the stationary point depends on a number of parameters, including the type of divalent metal cation, the concentration of the molecular crowding agent, and the type of beads.

### DETAILED DESCRIPTION OF THE INVENTION

### Definitions

In the context of the present invention, the term “buffer” is intended to mean a substance or mixture of substances that will maintain a relatively stable pH in an aqueous composition. Typically, when referring to a buffer by name, e.g. “Tris”, it refers to a mixture of the compound in question, e.g. Tris, and the corresponding acid/base, e.g. Tris-HCl.

When referring to molecular weights of polymers in the context of the present invention, it generally refers to number average molecular weight. As an example, when referring to polyethylene glycol, reference is made to the average number of repeating units in H—[OCH₂CH₂]ₙOH.

In the context of the present invention, the term “stationary point” is used in its mathematical sense, namely where the value of the derivative of one parameter as a function of another parameter is zero, i.e. where the first parameter reaches a (local) minimum or maximum value as a function of the second parameter. For example, in the context of the present invention, the cut-off value reaches a stationary point, in this case a minimum, as a function of the ratio of salt concentration to bead concentration.

In the context of the present invention, a “cut-off value” is a maximal or minimal value of a length of a DNA molecule (usually indicated by the number of bases), so that DNA molecules with larger or smaller chain length than this value are not supposed to be isolated with the size selective purification method of the present invention.

When referring to “isolating DNA molecules having a size above a certain cut-off value” in the context of the method of the present invention, the skilled person will understand that this does not necessarily refer to a 100% elimination of DNA molecules below the cut-off value. Thus, while the DNA molecules isolated in the method according to the present invention will predominantly be of a size above the cut-off value due to binding to the beads, a small quantity of DNA molecules having a size below the cut-off value may also bind to the beads.

### Beads

In order to separate the DNA from the DNA-containing sample, the DNA is desired to bind to the surface of the beads. Without being bound by a particular theory, the nucleobases of the DNA bind better to a negatively charged surface. The beads used in the method of the invention therefore preferably have a negatively charged surface. Different types of negatively charged surfaces of beads are known in the art. These include e.g. beads with a silicate surface, beads with a carboxylate-modified surface. Accordingly, in one embodiment of the invention, the negatively charged surface of the beads is a silicate or carboxylate-modified surface. In a further embodiment, the negatively charged surface of the beads is a carboxylate-modified surface. It is also possible to use beads with a surface having a mixture of both, a silicate and carboxylate-modified surface.

Depending on how the negatively charged surface is produced, it may not be necessary that the complete surface is covered with negative charges, but there may also be areas which are not negatively charged. The surface only needs to comprise enough negative charges to sufficiently bind the DNA. Consequently, in one embodiment, 80% or more of the particle surface should be covered with negative charges. In a further embodiment, 85% or more of the particle surfaces are covered with negative charges. In still a further embodiment, 90% or more of the particle surfaces are covered with negative charges. In yet a further embodiment, 95% or more of the particle surfaces are covered with negative charges. In another embodiment, 100% or more of the particle surfaces are covered with negative charges.

The beads with bound DNA used in the invention are supposed to be separated from the remaining composition containing the unbound, smaller DNA fragments. This separation may be accomplished in various ways, including methods utilizing centrifugal forces or gravity, optionally supported by vacuum or pressure, and methods utilizing magnetic forces, or any combination of those means. Accordingly, in one embodiment of the invention, the beads are magnetic beads. A particular kind of magnetic beads is known as solid-phase reverse immobilization (SPRI) beads. Thus, in another embodiment, the beads are SPRI beads. SPRI beads are commercially available and include Sera-Mag™ and Sera-Mag™ SpeedBeads (Sigma Aldrich), AMPure XP (Beckmann Coulter), PCRClean™ (Aline Biosciences), MagSi (AMSBIO), Axygen™ AxyPrep (Fischer Scientific), QIAseq (QIAGEN) and DNA IQ™ (Promega).

The beads, whether magnetic or non-magnetic, may also be contained in a column when the sample is added or the beads with already bound DNA may be added to a column for the purpose of separating the beads from the remaining composition. In case a liquid permeable closure of the column is used at its lower end, like a membrane, frit or similar, this allows binding, washing and/or eluting the DNA within the column in a flow through process.

### Molecular Crowding Agents

Molecular crowding agents (or “molecular crowders”) are molecules that, when used in sufficient concentration in a solution, can alter the properties of other molecules in that solution. Molecular crowders occupy volume and can concentrate other molecules in solution, illustratively by absorbing or locking up available water, thereby increasing the effective concentration of the other molecules. Molecular crowders can also affect the folding and binding of a variety of molecules. Molecular crowding is a well-known phenomenon (referring inter alia to Akabayov et al., Nature Communications, 4, 2013, article 1615) and e.g. has its own Wikipedia article (https://en.wikipedia.org/wiki/Macromolecular_crowding).

In the context of the present invention, without being bound by a particular theory, molecular crowders, such as (poly)ethylene glycol, are considered to affect the local concentration of divalent metal cations and/or DNA in the vicinity of the beads, thus affecting the cut-off value. It has surprisingly been observed that polyethylene glycols with different molecular weights but the same (weight) concentration percentage (w/v) affect the cut-off value in the same way. Thus, the effect seems to depend on the number of ethylene glycol monomers, which in turn points to a molecular crowding effect (see Example 10 and FIG. 11).

A number of molecular crowding agents are known from the prior art. These include hydrophilic polysaccharides, such as Ficolls, including Ficoll 70 and Ficoll 400, and dextran; sugars, such as sucrose; proteins, such as ovalbumin, BSA, and HSA; and polymers based on alkylene glycol, such as polyethylene glycol, and N-vinylpyrrolidone monomers. Hence, in one embodiment of the present invention, the molecular crowding agent is selected from the group consisting of hydrophilic polysaccharides, such as Ficolls, including Ficoll 70 and Ficoll 400, and dextran; sugars, such as sucrose; proteins, such as ovalbumin, BSA, and HSA; and polymers based on alkylene glycol, such as polyethylene glycol, and N-vinylpyrrolidone monomers. It has been found that molecular crowding agents based on alkylene glycol, such as ethylene glycol, and N-vinylpyrrolidone monomers provide particularly satisfactory results. Thus, in one embodiment, the molecular crowding agent is selected from the group consisting of an alkylene glycol, such as ethylene glycol, N-vinylpyrrolidone, oligomers thereof, polymers thereof, and mixtures of said monomers, oligomers, and polymers. In a further embodiment, the molecular crowding agent is selected from the group consisting of an alkylene glycol, such as ethylene glycol, oligomers thereof, polymers thereof, and mixtures of said monomers, oligomers, and polymers. In still a further embodiment, the molecular crowding agent is selected from the group consisting of ethylene glycol, propylene glycol, butylene glycol, pentylene glycol, oligomers thereof, polymers thereof, and mixtures of said monomers, oligomers, and polymers. In yet a further embodiment, the molecular crowding agent is selected from the group consisting of ethylene glycol, propylene glycol, butylene glycol, oligomers thereof, polymers thereof, and mixtures of said monomers, oligomers, and polymers. In still a further embodiment, the molecular crowding agent is selected from the group consisting of ethylene glycol, propylene glycol, oligomers thereof, polymers thereof, and mixtures of said monomers, oligomers, and polymers. In another embodiment, the molecular crowding agent is selected from the group consisting of ethylene glycol, diethylene glycol, triethylene glycol, tetraethylene glycol, polyethylene glycol (PEG), and mixtures thereof. In still another embodiment, the molecular crowding agent is PEG. In yet another embodiment, the molecular weight of the PEG is in the range of 200 to 35,000 Da, such as in the range of 1500 to 20,000 Da, e.g. in the range of 2000 to 18,000 Da, 3000 to 16,000 Da, 4000 to 15,000 Da, 5000 to 12000 Da, or 7000 to 9000 Da. In a further embodiment, the molecular crowding agent is PEG8000.

It may be preferred to use only polymers as molecular crowding agents. Thus, in one embodiment, the molecular crowding agent is selected from the group consisting of polyoxyalkylene, such as PEG, polyvinylpyrrolidone and mixtures thereof. In another embodiment, the molecular crowding agent is selected from the group consisting of polyethylene glycol (PEG), polypropylene glycol, polybutylene glycol, polypentylene glycol, and mixtures thereof. In still another embodiment, the molecular crowding agent is selected from the group consisting of polyethylene glycol (PEG), polypropylene glycol, polybutylene glycol, and mixtures thereof. In yet another embodiment, the molecular crowding agent is selected from the group consisting of polyethylene glycol (PEG), polypropylene glycol, and mixtures thereof.

### Divalent Metal Cations

Size selective binding to beads is traditionally carried out using sodium ions. However, Stortchevoi et al. among others have also used divalent metal cations, such as Mg²⁺ and Ca²⁺. It has been found that several different divalent metal cations work with the method of the present invention, which is therefore not considered limited to a particular divalent metal cation. Nevertheless, in one embodiment of the invention, the at least one divalent metal cation is selected from Mg, Ca, Sr, Co, Ni, Fe, Mn cations, and mixtures thereof. In a further embodiment, the at least one divalent metal cation is selected from Mg, Ca, Co cations, and mixtures thereof. In still a further embodiment, the at least one divalent metal cation is selected from Mg and Ca cations, as well as mixtures thereof.

In yet a further embodiment, the at least one divalent metal cation is a Mg cation. It is also possible to use a mixture of two or more divalent metal cations selected from Mg, Ca, Sr, Co, Ni, Fe, and Mn cations. In a preferred embodiment, only one divalent metal cation selected from Mg, Ca, Sr, Co, Ni, Fe, and Mn cations is used.

It is furthermore considered that the negative counterion to the divalent metal cation is not particularly limited, as long as the salt remains dissolved in the aqueous composition. The counterion may shift the cut-off value slightly but is not expected to change the way the divalent metal cation functions in the method of the invention. In one embodiment, the counterion of the divalent metal cation is a divalent or monovalent anion. In a further embodiment, the counterion of the divalent metal cation is a monovalent anion. In another embodiment, the counterion of the divalent cation is selected from the group consisting of halide, such as fluoride, chloride, bromide, or iodide, sulfate, nitrate, carbonate, hydroxide, acetate, and mixtures thereof. In still another embodiment, the counterion of the divalent cation is selected from the group consisting of halide, such as fluoride, chloride, bromide, or iodide, sulfate, nitrate, acetate, and mixtures thereof. In a further embodiment, the counterion of the divalent cation is selected from the group consisting of fluoride, chloride, bromide, and iodide. In still another embodiment, the counterion is selected from fluoride, chloride, and bromide. In yet another embodiment, the counterion is selected from chloride and bromide. In a further embodiment, the counterion is chloride.

### Buffers

The method of the present invention may be carried out in the presence or absence of a buffer in the aqueous composition. The buffer serves to stabilize pH and thus creates a controlled environment and may also contribute to the overall ionic strength of the aqueous composition. In principle, any buffer typically used in biological applications involving DNA, such as Tris-HCl, may be used. In one embodiment, the buffer is present in the aqueous composition and provides a pH in the range of 5-9. In another embodiment, the buffer is present in the aqueous composition and provides a pH in the range of 7-9. In a further embodiment, the buffer is present in the aqueous composition and the buffer is a zwitterion at the buffer pH.

### DNA-Containing Sample

The DNA-containing sample comprises DNA molecules of different sizes (lengths). The DNA-containing sample may comprise single-stranded and/or double stranded DNA. The method according to the present invention allows size selection of single stranded as well as of double-stranded DNA. In one embodiment, the DNA molecules of the DNA-containing sample are double-stranded DNA molecules. In another embodiment, the DNA molecules of the DNA-containing sample are linear, double-stranded DNA molecules.

The DNA-containing sample can be of various origins, including biological samples and artificial samples that were obtained during nucleic acid processing. According to one embodiment, the DNA-containing sample is a sample of extracted DNA or extracted DNA that has been further processed, e.g. by shearing or by way of an enzymatic reaction. According to one embodiment, the DNA-containing sample was obtained after an enzymatic reaction. Exemplary enzymatic reactions that provide DNA-containing samples that can be processed using the methods of the invention include but are not limited to amplification reactions, ligase reactions, in particular adapter ligation reactions and polynucleotide, e.g. poly A, tailing reactions. According to one embodiment, the DNA-containing sample comprises fragmented DNA, e.g. sheared DNA. According to one embodiment, the DNA-containing sample comprises sheared genomic DNA or sheared cDNA. Thus, according to one embodiment the DNA-containing sample is a solution resulting from a size shearing procedure. Such DNA-containing sample comprises DNA fragments of different sizes.

Said fragmented DNA can be end-repaired and/or internally repaired. Thus, according to one embodiment, the DNA-containing sample comprises linear DNA fragments of different sizes. According to one embodiment, the DNA-containing sample was obtained during the preparation of a sequencing library, in particular during preparation of a next generation sequencing library. According to one embodiment, the DNA-containing sample comprises amplification products, e.g. PCR products. Thus, according to one embodiment, the DNA-containing sample is a solution resulting from an amplification procedure, in particular resulting from a PCR amplification. According to one embodiment, the DNA-containing sample is an adapter ligation sample that was obtained as a result of an adapter ligation step. According to a preferred embodiment, the DNA-containing sample is an adapter ligation sample which comprises (i) double-stranded DNA molecules that are flanked 5′ and/or 3′ by adapters, (ii) adapter monomers and (iii) adapter-adapter ligation products such as e.g. adapter dimers. Furthermore, the DNA-containing sample may comprise additional contaminating components such as e.g. mono, oligo- and/or polynucleotides and proteins such as enzymes that are e.g. still present in the DNA-containing sample from previous enzymatic sequencing library processing steps.

### Contacting, Separation, Washing, and Elution of the Beads

Contacting the DNA-containing sample with the aqueous composition in step a) to provide a binding mixture and binding of the DNA molecules to the beads may be performed simultaneously or sequentially. According to one embodiment, the DNA-containing sample is contacted with the aqueous composition and the resulting binding mixture is then contacted with the beads. The beads, the aqueous composition and the DNA-containing sample can be added in any order. E.g. it is within the scope of the present invention to first provide the beads and the aqueous composition and then add the DNA-containing sample or to first provide the DNA-containing sample, the beads and then add the aqueous composition. Preferably, the aqueous composition is mixed with the DNA-containing sample to provide a binding mixture, to which the beads are then added.

At the end of step a), predominantly DNA molecules having a size above the cut-off value are bound to the beads. Thus, while the DNA molecules isolated in the method according to the present invention will predominantly be of a size above the cut-off value due to binding to the beads, a small quantity of DNA molecules having a size below the cut-off value may also bind to the beads. In a preferred embodiment, the amount of DNA having a size below the cut-off value that binds to the beads is 10% or less, preferred 5% or less, more preferred 3% or less, and most preferred 2% or less.

In step b), the DNA that is bound to the beads is separated from the remaining sample. Thereby, the adsorbed DNA having a size above the cut-off value is separated from unbound DNA molecules and optionally other contaminants and impurities present in the sample. Suitable separation methods are well known in the art and the appropriate separation technique also depends on the type of beads used. This process can be assisted e.g. by centrifugation. The beads may also be collected in any kind of filter or filter column as is known in the art and the separation may then be supported by applying a vacuum or pressure. When using e.g. silica beads, the beads can be collected by sedimentation which can be assisted by centrifugation. If magnetic beads are used, magnetic separation may be applied in addition to the aforementioned methods.

Depending on the information that is desired from the sample, the DNA with a size below the cut-off value remaining in the sample, after the beads with the bound target DNA having a size above the cut-off value are removed, may be further isolated with standard purification systems known in the art like e.g. QIAquick (QIAGEN GmbH). If the separated DNA having a size below the cut-off value is of no further interest, the remaining sample may also be discarded after the beads with the target DNA having a size above the cut-off value bound thereon have been separated.

In optional step c), the bound DNA is washed. Here, one or more washing steps can be performed. Even though this step is optional, it is preferably performed in order to efficiently remove unbound components and impurities such as e.g. nucleotides and enzymes from previous reactions. This is particularly suitable if the DNA-containing sample was obtained during the preparation of a sequencing library. Furthermore, washing steps are also suitable to remove traces of the salt used during binding, if it could interfere with the intended downstream process.

Thus, according to one embodiment, one or more washing steps are performed in step c) in order to further purify the bound DNA molecules. For this purpose, common washing solutions may be used. A suitable washing solution removes impurities but preferably not the DNA that is bound to the beads or at least bound DNA is only removed in acceptable amounts to still ensure a sufficient yield of target DNA.

According to one embodiment, the solution used for washing comprises at least one chaotropic salt and/or at least one alcohol. Chaotropic salts that can be used in the washing solutions include but are not limited to guanidinium hydrochloride, guanidinium thiocyanate, guanidinium isothiocyanate and sodium iodide or other chaotropic salts. As alcohol, short chained branched or unbranched alcohols with preferably one to 5 carbon atoms can be used for washing, respectively can be used in the washing solution. Also mixtures of alcohols can be used. Suitable alcohols include but are not limited to methanol, ethanol, propanol, isopropanol and butanol. Preferably, isopropanol and/or ethanol are used in the washing solution.

A further suitable washing solution which can be used alternatively or also in addition to the washing solutions described above comprises an alcohol and a buffering agent. Suitable alcohols and buffering agents such as biological buffers are described above. Preferably, isopropanol or ethanol, most preferred ethanol is used for this second washing step. Preferably, ethanol is used in a concentration of from 30% (v/v) to 80% (v/v), such as 40% (v/v) to 70% (v/v), e.g. around 50% (v/v).

A further suitable washing solution which can be used alternatively or optionally also in addition to the washing solutions described above comprises an alcohol but no salt. This allows to wash away salts. Preferably, isopropanol or ethanol, most preferred ethanol is used for washing. Preferably, the alcohol is used in a concentration of from 20% (v/v) to 80% (v/v), such as 30% (v/v) to 70% (v/v), 40% (v/v) to 60% (v/v), e.g. around 50% (v/V).

Residual alcohol that may be present after the washing step in case an alcohol containing washing solution was used, can be removed e.g. by air drying (suitable when working with magnetic beads) or by an additional centrifugation step in particular if using non-magnetic beads. Respective methods and procedures are well-known in the art and thus, do not need any further description here.

Another suitable washing solution, which can be used alternatively or optionally also in addition to the washing solutions described above, comprises a molecular crowding agent, such as PEG, as described above. The molecular crowding agent can be used optionally in combination with a salt, wherein the cations and anions used are not limited to the ions used in the aqueous composition according to step a) of the invention. The molecular crowding agent and the ions are used in concentrations which sustain binding conditions for all molecules of desired fragment length. This may either be a concentration which sustains binding conditions for all bound fragments independent of their fragment length, or it can be a concentration where fragments below a certain length are washed away from the beads, thus supporting the size selective purification process of the invention.

In optional step d), one or more elution steps are performed in order to elute the purified size selected DNA. However, the bound DNA may also be processed while being bound to the beads, depending on the intended downstream application or the intended use of the DNA, respectively.

However, it is preferred to elute the DNA. Here, basically any elution solution can be used which effects desorption of the bound DNA from the binding matrix. Classical elution solutions known to effectively elute DNA from a bead surface include but are not limited to water, elution buffers, such as TE-buffer (10 mM Tris-Cl, 1 mM EDTA, pH 8.0), EB Buffer (10 mM Tris-Cl, pH 8.5) or AE Buffer (10 mM Tris-Cl, 0.5 mM EDTA, pH 9.0) (all QIAGEN, Germany) and low-salt solutions which have a salt concentration of 150 mM or less, preferably 100 mM or less, more preferred 75 mM or less, 50 mM or less, 25 mM or less, 20 mM or less, 15 mM or less, 10 mM or less or are salt-free. The elution solution may e.g. comprise a buffering agent, in particular may comprise a biological buffer such as Tris, MOPS, HEPES, MES, BIS-TRIS, and others. The buffering agent may be present in a concentration of 150 mM or less, preferably 100 mM or less, more preferred 75 mM or less, 50 mM or less, 25 mM or less, 20 mM or less, 15 mM or less or 10 mM or less. According to one embodiment, the elution buffer has a pH value that is selected from pH 6.5 to pH 11, pH 7 to pH 10, pH 8 to pH 9.5. Elution can be assisted by heating and/or shaking.

The elution buffer may also contain a complexing agent like EDTA or EGTA in low concentrations to inhibit contaminating DNase by complexing divalent cations like Mg²⁺, Ca²⁺, Zn²⁺ and others which are essential cofactors for these enzymes.

Furthermore, it is also within the scope of the present invention to repeat the elution step in order to ensure that the bound DNA is efficiently released from the binding matrix.

### Ratio of Concentration of Salt to the Concentration of Beads

The prior art observed that increasing salt to bead ratios led to decreasing cut-off values with a high gradient leading to the potential for high sensitivity of changes in the cut-off value e.g. due to pipetting errors. It furthermore meant a relatively low upper limit to the cut-off values being achievable of up to 1.5 to 2 kbp. The inventors of the present invention have surprisingly observed that this tendency has a stationary point upon increasing the salt to bead concentration ratio further, after which point the cut-off value increases with increasing salt to bead concentration ratio. It has further been observed that high cut-off values, such as ≥3 kbp, in one embodiment ≥4 kbp, in a further embodiment ≥5, in still a further embodiment ≥7 kbp, in yet a further embodiment ≥8 kbp, and even allowing ≥10 kbp, hitherto impossible to achieve with the prior art methods may be achieved with the present invention. Hence, the method of the present invention is suitable for preparing DNA samples for third generation sequencing such as long-read sequencing.

Thus, the ratio of the concentration of the at least one salt to the concentration of beads in the aqueous composition is at a value where an increase in the ratio leads to an increase in said cut-off value, i.e. where the derivative of the cut-off value as a function of the salt to bead concentration ratio is positive. Furthermore, the amplitude or absolute value of this positive derivative is smaller than the amplitude or absolute value of the negative derivative at salt to bead concentration ratios below the stationary point. This principle is illustrated in FIG. 1, which is based on the lower panel of FIG. 4. The red line is not the exact curve representing the underlying mathematical function but serves as an illustration of the cut-off value as a function of the Mg²⁺ concentration (increasing from left to right with constant bead concentration). The principle is also illustrated in FIG. 12.

As demonstrated in the Examples below, the exact location of the stationary point and, thereby, also of the cut-off value depends on and may be influenced by additional parameters, including the type of the at least one divalent metal cation, the concentration and type of the molecular crowding agent, the buffer, and the type of beads. The effect of those components, either alone or in any combination, may advantageously be used to further optimize or finetune the position of the stationary point, the position of a specific cut-off, and/or the derivative of the cut-off value as a function of the salt to bead concentration ratio. Thus, the stationary point may vary depending on the parameters of the chosen system of components. However, the common feature is that all those combinations have a stationary point when plotting the increase in the ratio of the concentration of salt to the concentration of beads in relation to the cut-off value, above which stationary point the method according to the invention is carried out. In one embodiment, the ratio of the concentration of salt to the concentration of beads in the aqueous composition is increased at least 10% compared to the ratio where a small increase neither leads to an increase nor a decrease in cut-off value. In a further embodiment, the ratio of the concentration of salt to the concentration of beads in the aqueous composition is increased at least 15% compared to the ratio where a small increase neither leads to an increase nor a decrease in cut-off value. In still a further embodiment, the ratio of the concentration of salt to the concentration of beads in the aqueous composition is increased at least 20% compared to the ratio where a small increase neither leads to an increase nor a decrease in cut-off value. In yet a further embodiment, the ratio of the concentration of salt to the concentration of beads in the aqueous composition is increased at least 25% compared to the ratio where a small increase neither leads to an increase nor a decrease in cut-off value. In another embodiment, the ratio of the concentration of salt to the concentration of beads in the aqueous composition is increased at least 30% compared to the ratio where a small increase neither leads to an increase nor a decrease in cut-off value. In still another embodiment, the ratio of the concentration of salt to the concentration of beads in the aqueous composition is increased at least 35% compared to the ratio where a small increase neither leads to an increase nor a decrease in cut-off value. In yet another embodiment, the ratio of the concentration of salt to the concentration of beads in the aqueous composition is increased at least 40% compared to the ratio where a small increase neither leads to an increase nor a decrease in cut-off value. If more than one divalent cation is used in the salt, the above indicated salt concentrations refer to the total amount of salt that is used. Preferably, the salt contains only one divalent metal cation. It has been seen that the valence of the counter anion in the salt may have an impact on the amplitude or absolute value of the derivative of the cut-off value as a function of the salt to bead concentration ratio. Thus, the steeper the gradient of the red line in FIG. 1, the more sensitive the system against changes in the cut-off value e.g. due to pipetting errors. Thus, in one embodiment, the salt does not contain any divalent counter anions and in a further embodiment, it does not contain a sulfate anion.

In one embodiment, the total concentration of the divalent metal cation is 50 mM or higher. In another embodiment, the total concentration of the divalent cation is 100 mM or higher. In still another embodiment, the total concentration of the divalent cation is 150 mM or higher. In a further embodiment, the total concentration of the divalent metal cation is in the range of 50 mM to 1000 mM. In still a further embodiment, the total concentration of the divalent cation is in the range of 100 mM to 800 mM. In yet a further embodiment, the total concentration of the divalent cation is in the range of 125 mM to 700 mM. In still a further embodiment, the total concentration of the divalent cation is in the range of 150 mM to 500 mM. In yet a further embodiment, the total concentration of the divalent cation is in the range of 200 mM to 400 mM. In another embodiment, the divalent metal cation is Mg and the concentration is in the range of 100 mM to 500 mM. In still another embodiment, the divalent metal cation is Mg and the concentration is in the range of 125 mM to 400 mM. In yet another embodiment, the divalent metal cation is Mg and the concentration is in the range of 150 mM to 350 mM.

If two or more divalent cations are used together, the above indicated concentrations refer to the total amount of all divalent metal cations that are used. Preferably, only one divalent metal cation is used.

As observed i.a. for Co²⁺ and Mn²⁺, the method of the invention also provides an upper cut-off value in addition to the lower cut-off value, i.e. the bound DNA has a molecular size between the two cut-off values. Thus, in one embodiment, the method of the invention is a method for isolating DNA molecules having a size between two different cut-off values from a DNA-containing sample. It is particularly advantageous in this embodiment that DNA having a size smaller than the lower cut-off value and DNA having a size above the upper cut-off value can be removed in one step resulting only in the desired middle-sized fraction by choosing the corresponding conditions. In a further embodiment, the method of the invention is a method for isolating DNA molecules having a size between two different cut-off values from a DNA-containing sample, wherein the divalent metal cation is selected from Co and Mn, and mixtures thereof.

### BRIEF DESCRIPTION OF THE FIGURES

FIG. 1

Illustrative figure based on the lower panel in FIG. 4, showing the principle of the invention.

FIG. 2

Size selection depending on the amount of magnetic beads.

FIG. 3

Size selection depending on the amount of magnetic beads.

FIG. 4

Size selection depending on the Mg²⁺ concentration: upper row: GelPilot® 1kbp plus ladder (QIAGEN, Germany); bottom row: HMW DNA; lane 22 represents the original sample material.

FIG. 5

PFGE of size selected HMW DNA. The white line indicates an increasing cut-off from lane 8 to 17.

FIG. 6

Size selection with different divalent cations (Co²⁺, Mn²⁺, Ca²⁺).

FIG. 7

Size selection with different concentrations of Ca²⁺ (upper image) and Mg²⁺ (lower image). The lower image is from Example 3 (numbers 3-18 in Example 3 correspond to 1-16 in Example 5).

FIG. 8

Size selection with different concentrations of PEG8000.

FIG. 9

Size selection with different amounts of DNA.

FIG. 10

Size selection up to 40kbp with different concentrations of Mg²⁺ (left) and virtual gel for better visualization of the cut-off of about 40 kbp (right).

FIG. 11

Size selection with different concentrations of PEG. The red frame (lane 1) shows the amount of PEG8000 used in the other examples.

FIG. 12

N50 values of sequenced DNA after size selection with different concentrations of Mg²⁺.

FIG. 13

Tape Station (Agilent) analysis of DNA isolated with the Power Soil Pro Kit (A1: DNA size marker; B1-D1: w/o size selection; E1-G1: size selection according to the invention; H1-E2: AMPure size selection with 0.4× and 0.6× sample of AMPure bead suspension, respectively).

FIG. 14

N50 values of sequenced DNA isolated with the PowerSoil Pro Kit w/o and after size selection.

### SPECIFIC EMBODIMENTS

1. A method for isolating DNA molecules having a size above a certain cut-off value from a DNA-containing sample, comprising
   a) contacting the sample with an aqueous composition comprising beads with a negatively charged surface, a molecular crowding agent, a dissolved salt comprising at least one divalent metal cation, and optionally a buffer for a time sufficient to bind DNA to the surface of the beads;
   b) separating the beads with bound DNA from the remaining composition;
   c) optionally washing the beads; and
   d) optionally eluting the bound DNA from the beads;
   wherein the ratio of the concentration of salt to the concentration of beads in the aqueous composition in step a) is at a value where an increase in the ratio leads to an increase in said cut-off value.
2. The method according to embodiment 1, wherein the negatively charged surface of the beads is a silicate or carboxylate-modified surface.
3. The method according to embodiment 1 or 2, wherein the beads are magnetic beads.
4. The method according to any one of the preceding embodiments, wherein the beads are solid-phase reversible immobilization (SPRI) beads.
5. The method according to embodiment 4, wherein the beads are SPRI beads with a carboxylate-modified surface.
6. The method according to any one of the preceding embodiments, wherein the molecular crowding agent is selected from the group consisting of an alkylene glycol, such as ethylene glycol, N-vinylpyrrolidone, oligomers thereof, polymers thereof, and mixtures of said monomers, oligomers, and polymers.
7. The method according to embodiment 6, wherein the molecular crowding agent is selected from the group consisting of an alkylene glycol, such as ethylene glycol, oligomers thereof, polymers thereof, and mixtures of said monomers, oligomers, and polymers.
8. The method according to embodiment 6, wherein the molecular crowding agent is selected from the group consisting of ethylene glycol, propylene glycol, butylene glycol, pentylene glycol, oligomers thereof, polymers thereof, and mixtures of said monomers, oligomers, and polymers.
9. The method according to embodiment 6, wherein the molecular crowding agent is selected from the group consisting of ethylene glycol, propylene glycol, butylene glycol, oligomers thereof, polymers thereof, and mixtures of said monomers, oligomers, and polymers.
10. The method according to embodiment 6, wherein the molecular crowding agent is selected from the group consisting of ethylene glycol, propylene glycol, oligomers thereof, polymers thereof, and mixtures of said monomers, oligomers, and polymers.
11. The method according to embodiment 6, wherein the molecular crowding agent is selected from the group consisting of ethylene glycol, diethylene glycol, triethylene glycol, tetraethylene glycol, polyethylene glycol (PEG), and mixtures thereof.
12. The method according to embodiment 6, wherein the molecular crowding agent is PEG.
13. The method according to embodiment 12, wherein the molecular weight of the PEG is in the range of 200 to 35,000 Da, such as in the range of 1500 to 20,000 Da, e.g. in the range 5000 to 12000 Da.
14. The method according to embodiment 13, wherein the molecular crowding agent is PEG8000.
15. The method according to embodiment 6, wherein the molecular crowding agent is selected from the group consisting of polyoxyalkylene, such as PEG, polyvinylpyrrolidone and mixtures thereof.
16. The method according to embodiment 6, wherein the molecular crowding agent is selected from the group consisting of polyethylene glycol (PEG), polypropylene glycol, polybutylene glycol, polypentylene glycol, and mixtures thereof.
17. The method according to embodiment 6, wherein the molecular crowding agent is selected from the group consisting of polyethylene glycol (PEG), polypropylene glycol, polybutylene glycol, and mixtures thereof.
18. The method according to embodiment 6, wherein the molecular crowding agent is selected from the group consisting of polyethylene glycol (PEG), polypropylene glycol, and mixtures thereof.
19. The method according to any one of the preceding embodiments, wherein the concentration of the molecular crowding agent in the aqueous composition is in the range 0.1% to 3% (w/v), such as 0.5% to 2% (w/v), e.g. 0.75 to 1.25% (w/V).
20. The method according to any one of the preceding embodiments, wherein the at least one divalent metal cation is selected from Mg, Ca, Sr, Co, Ni, Fe, and Mn cations, and mixtures thereof.
21. The method according to embodiment 20, wherein the at least one divalent metal cation is selected from Mg, Ca, and Co cations, and mixtures thereof.
22. The method according to embodiment 20, wherein the at least one divalent metal cation is selected from Mg and Ca cations, and mixtures thereof.
23. The method according to embodiment 20, wherein the at least one divalent metal cation is a Mg cation.
24. The method according to any one of the preceding embodiments, wherein the ratio of the concentration of salt to the concentration of beads in the aqueous composition is increased at least 10% compared to the ratio where a small increase neither leads to an increase nor a decrease in cut-off value.
25. The method according to any one of the preceding embodiments, wherein the total concentration of the divalent metal cation is in the range 50 mM to 1000 mM.
26. The method according to embodiment 25, wherein the total concentration of the divalent cation is in the range 75 mM to 800 mM.
27. The method according to embodiment 25, wherein the total concentration of the divalent cation is in the range 100 mM to 700 mM.
28. The method according to embodiment 25, wherein the total concentration of the divalent cation is in the range 120 mM to 600 mM.
29. The method according to embodiment 25, wherein the total concentration of the divalent cation is in the range 150 mM to 500 mM.
30. The method according to any one of the preceding embodiments, wherein the counterion of the divalent cation is selected from the group consisting of halide, such as fluoride, chloride, bromide, or iodide, sulfate, nitrate, carbonate, hydroxide, acetate, and mixtures thereof.
31. The method according to embodiment 30, wherein the counterion of the divalent cation is selected from the group consisting of halide, such as fluoride, chloride, bromide, or iodide, sulfate, nitrate, acetate, and mixtures thereof.
32. The method according to embodiment 30, wherein the counterion of the divalent cation is selected from the group consisting of fluoride, chloride, bromide, and iodide.
33. The method according to embodiment 30, wherein the counterion is selected from fluoride, chloride, and bromide.
34. The method according to embodiment 30, wherein the counterion is selected from chloride and bromide.
35. The method according to embodiment 30, wherein the counterion is chloride.
36. The method according to any one of the preceding embodiments, wherein the buffer is present in the aqueous composition and provides a pH in the range 5-9.
37. The method according to embodiment 36, wherein the buffer is present in the aqueous composition and provides a pH in the range 7-9.
38. The method according to any one of the preceding embodiments, wherein the buffer is present in the aqueous composition and the buffer is a zwitterion at the buffer pH.
39. The method according to any one of the preceding embodiments, wherein the DNA bound to the beads has a size between a lower cut-off value and an upper cut-off value.
40. The method according to embodiment 39, wherein the at least one divalent metal cation is selected from Co and Mn, and mixtures thereof.

### EXAMPLES

### Abbreviations

HMW high molecular weight
M molarity
ONT Oxford Nanopore Technology
PEG polyethylene glycol
PFGE pulse field gel electrophoresis
SPRI solid phase reversible immobilization

### Experimental Protocol

All experiments were done according to the following protocol:

The GelPilot® 1kbp Plus Ladder (QIAGEN, Hilden, Germany) was purified to get rid of the contained dyes according to the QIAquick PCR clean-up (QIAGEN, Hilden, Germany) protocol.
1.2 μg purified GelPilot® 1kbp Plus Ladder was mixed with divalent cation solution to a total volume of 50 μl
25 μl Bead suspension (which includes PEG and Tris) was added and the volume adjusted with RNase free water to 50 μl
The resulting mixture (100 μl total) was mixed by pipetting up and down and afterwards incubated for 10 min at ambient temperature.
The beads were collected at a magnet, the supernatant was discarded.
The beads were kept attached to the magnet and the pellet was washed with 70% ethanol
The beads were air dried for 5 min
DNA was eluted by adding 30 μl Buffer EB (QIAGEN).
The beads were collected and the eluate was transferred into a new tube
12 μl of the eluate+3 μl loading buffer were used for agarose gel (1%) electrophoresis

The bead concentrations are given in % of 100% stock solution (50 mg/ml).

The beads were washed twice with water and once with the appropriate binding buffer containing PEG and Tris, before being resuspended in the appropriate binding buffer containing PEG and Tris.

Size Selection reactions were prepared according to the following protocol:

### Example 1—Effect of Bead/Divalent Cation Concentration

Bead Suspension: 2% (w/v) PEG8000; 20 mM Tris pH, pH 7.5; Sera-Mag® Magnetic carboxylate-modified beads with variable concentrations
Divalent cation solution: 150 mM or 300 mM MgCl₂

TABLE 1
Reaction set-ups with Mg²⁺ and bead concentrations (6.1:
repeat of 2a with doubled amount of DNA (2.4 μg))

| | MgCl₂ [M] | Beads (%) |
|---|---|---|
| 1a | 0.15 | 100 |
| 1b | 0.15 | 75 |
| 1c | 0.15 | 50 |
| 1d | 0.15 | 25 |
| 2a | 0.3 | 100 |
| 2b | 0.3 | 75 |
| 2c | 0.3 | 50 |
| 2d | 0.3 | 25 |
| 6.1 | 0.3 | 100 |

### Results

The size selection results are shown in FIG. 2. This experiment demonstrates a clear relationship between the amount of beads used and the cut-off (meaning the smallest fragment still bound and isolated):

Reducing the amount of beads corresponds to increasing the molarity of Mg²⁺/bead, which in turn increases the cut-off. This effect can also be achieved by increasing the amount of Mg²⁺ at constant amounts of beads (compare Examples 1 and 2).

In addition, the effect is independent of the DNA concentration as demonstrated when comparing 6.1 and 2a.

### Example 2—Effect of Bead/Divalent Cation Concentration

Bead Suspension: 2% (w/v) PEG8000; 20 mM Tris pH 7.5; Sera-Mag® Magnetic carboxylate-modified beads with variable concentrations
Divalent cation solution: 150 mM MgCl₂

In this experiment all parameters were kept constant, only the relative amount of beads was further decreased equivalent to a stepwise increase of the Mg²⁺ concentration relative to the beads.

TABLE 2
Reaction set-ups with bead concentrations

| | Beads (%) |
|---|---|
| 1 | 200 |
| 2 | 175 |
| 3 | 150 |
| 4 | 125 |
| 5 | 100 |
| 6 | 75 |
| 7 | 50 |
| 8 | 25 |
| 9 | 10 |
| 10 | 5 |
| 11 | 1 |

### Results

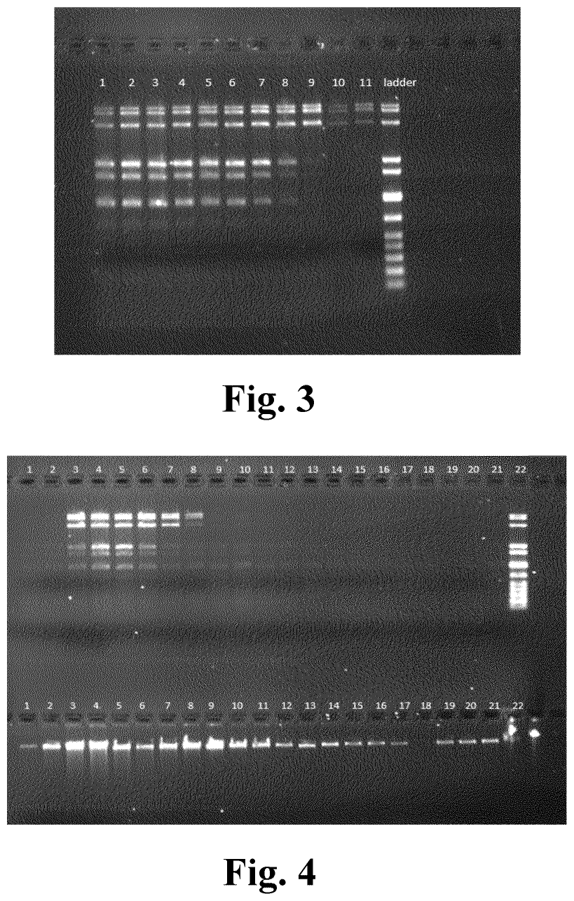

The size selection results are shown in FIG. 3. This experiment confirms the results from Example 1, showing a size-dependent binding with decreasing amounts of beads equivalent to an increasing ratio of Mg²⁺/beads. Considering lanes 8 and 9, there is no loss in the larger fragments even if the smaller fragments vanish more and more. This indicates a true size-selective binding.

### Example 3—Effect of Divalent Cation Concentration

To confirm the hypothesis of Mg²⁺/bead ratio, in this experiment only the Mg²⁺ concentrations were varied.
Bead Suspension: 2% (w/v) PEG8000; 20 mM Tris pH 7.5, Sera-Mag® Magnetic carboxylate-modified beads (5% w/V)
Divalent cation solution: variable MgCl₂.

TABLE 3
Reaction set-ups with Mg²⁺ concentrations.

| No. | 1 | 2 | 3 | 4 | 5 | 6 | 7 | 8 | 9 | 10 |
|---|---|---|---|---|---|---|---|---|---|---|
| c(Mg2+) [M] | 0.001 | 0.05 | 0.1 | 0.15 | 0.2 | 0.25 | 0.3 | 0.35 | 0.4 | 0.45 |

| No. | 11 | 12 | 13 | 14 | 15 | 16 | 17 | 18 | 19 | 20 | 21 |
|---|---|---|---|---|---|---|---|---|---|---|---|
| c(Mg2+) [M] | 0.5 | 0.55 | 0.6 | 0.65 | 0.7 | 0.75 | 0.8 | 0.85 | 0.9 | 0.95 | 1 |

Besides the GelPilot® 1kbp plus Ladder, a HMW DNA isolated with the MagAttract HMW DNA Kit (QIAGEN, Hilden, Germany) was used as sample material.

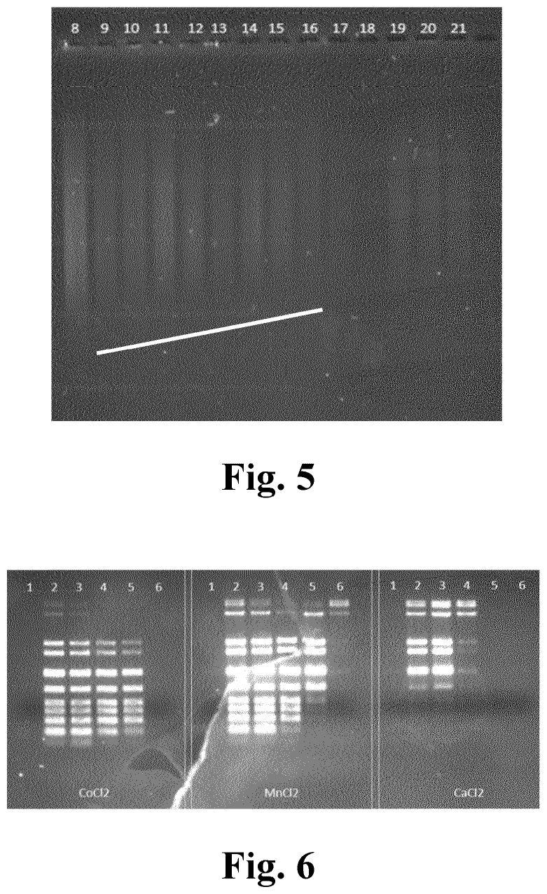

For a better estimation of the size selective binding, HMW samples 8 to 21 shown in FIG. 4 were also analyzed by PFGE (FIG. 5).

### Results

The results from this example confirm the previous data. Instead of changing the Mg²⁺/bead ratio by reducing the amount of beads, increasing the Mg²⁺ concentration has the same effect (as can be seen in FIG. 4, upper row).

In FIG. 4, lower row, a decrease in signal intensity could be observed. There are two assumed reasons for this:
1) weakened binding with increasing Mg²⁺ concentration and therefore loss of DNA
2) more loss of smaller DNA fragments with increasing Mg²⁺ concentrations because the larger fragments do not separate in a standard agarose gel forming one band together.

In a PFGE it became obvious that there is indeed a size selection as indicated by the reference line in FIG. 5, especially from lane 8 to 17.

### Example 4—Different Divalent Cations

To see if the effect observed in the previous examples is a special effect of Mg²⁺ or a general feature of divalent cations, other alkaline earth and transition metals were tested: Co²⁺, Mn²⁺, Ca²⁺ (chloride each).
Bead Suspension: 2% (w/v) PEG8000; 20 mM Tris pH 7.5, Sera-Mag® Magnetic carboxylate-modified beads (50% w/v)
Divalent cation solution: variable MetalCl₂.

TABLE 4
Reaction set-ups with Metal²⁺ concentrations

| No. | Salt (M) |
|---|---|
| 1 | 0.001 |
| 2 | 0.1 |
| 3 | 0.2 |
| 4 | 0.4 |
| 5 | 0.6 |
| 6 | 1 |

### Results

The data are shown in FIG. 6 and clearly demonstrate that a size selection according to the invention—a higher concentration of cations with constant amount of beads leads to higher cut-offs—works with divalent cations in general, independent of the metal (main group element (Mg²⁺, Ca²⁺) or transition metal element (Co²⁺, Mn²⁺)).

In addition, with the transition metals a two-sided removal of fragments is possible as visible in lanes 4 and 5 with Co²⁺ and lane 4 with Mn²⁺.

### Example 5—Comparison of Mg²⁺ and Ca²⁺

For the comparison, Ca²⁺ was tested and compared to the results obtained in Example 3.
Bead Suspension: 2% (w/v) PEG8000; 20 mM Tris pH 7.5; Sera-Mag® Magnetic carboxylate-modified beads (50% w/V)
Divalent cation solution: variable Mg²⁺ and Ca²⁺

TABLE 5
Reaction set-ups with different Metal²⁺ concentrations

| No. | 1 | 2 | 3 | 4 | 5 | 6 | 7 | 8 |
|---|---|---|---|---|---|---|---|---|
| c(Ca²⁺) [M] | 0.1 | 0.15 | 0.2 | 0.25 | 0.3 | 0.35 | 0.4 | 0.45 |
| No. | 9 | 10 | 11 | 12 | 13 | 14 | 15 | 16 |
| c(Ca²⁺) [M] | 0.5 | 0.55 | 0.6 | 0.65 | 0.7 | 0.75 | 0.8 | 0.85 |

### Results

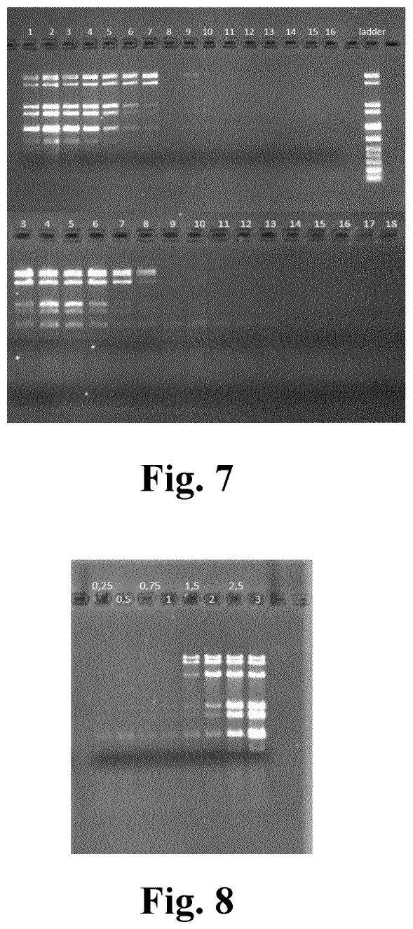

The results are shown in FIG. 7 with the upper panel showing the results for Ca²⁺ and the lower panel showing the results for Mg²⁺ taken from Example 3 (for a better comparison regarding the used concentrations, lanes 3-18 in FIG. 4 of Example 3 correspond to lanes 1-16 here).

Both Mg²⁺ and Ca²⁺ showed higher cut-offs with increasing cation concentration at constant amount of beads. The Ca²⁺ change seems to be slower than with Mg²⁺. This means that a Ca²⁺ system may be even more robust to slight changes in cation concentration, for example due to pipetting errors, than Mg²⁺.

### Example 6—Effect of the Buffer

In this example the influence of other components of the binding composition on the cut-off was investigated. Tris-concentrations were varied at constant pH and constant Mg²⁺ concentration, and the effect of ionic strength was tested by adding sodium ions at different concentrations in the second part.
Bead Suspension: 2% (w/v) PEG8000; variable Tris pH 7.5; Sera-Mag® Magnetic carboxylate-modified beads (50% w/v)
Divalent cation solution: 300 mM Mg²⁺

Tables 6 and 7: Reaction set-ups with different
Tris-HCl and NaCl concentrations.

| No. | Tris-HCl [M] |
|---|---|
| 1 | 0.1 |
| 2 | 0.07 |
| 3 | 0.05 |
| 4 | 0.03 |
| 5 | 0.02 |
| 6 | 0.015 |
| 7 | 0.01 |

| No. | Tris-HCl | NaCl | pH |
|---|---|---|---|
| 1 | 0.1M | — | 7.5 |
| 2 | 0.01M | — | 7.5 |
| 3 | 0.01M | 0.1M | 7.5 |
| 4 | 0.01M | 0.01M | 7.5 |
| 5 | — | 0.01M | 4 |

### Results

The compositions at constant Mg²⁺ concentration and varying Tris-HCl concentration according to Table 6 with different buffer capacities at the same pH all provide an adjustable cut-off value as seen in the previous examples.

Furthermore, comparison of compositions 4 and 5 in Table 7 finds that pH does not have an effect on the cut off, as these two compositions have the same cut-off value.

### Example 7—Effect of the Concentration of the Viscosity-Enhancing Compound

It was investigated whether the concentration of the viscosity-enhancing compound, in this case PEG8000, has an effect on the cut-off value.
Bead Suspension: variable PEG8000; 20 mM Tris pH 7.5; Sera-Mag® Magnetic carboxylate-modified beads (50% w/V)
Divalent cation solution: 400 mM Mg²⁺

TABLE 8
Reaction set-ups with different PEG8000 concentrations.

| No. | PEG [%(w/v)] |
|---|---|
| 1 | 0.25 |
| 2 | 0.5 |
| 3 | 0.75 |
| 4 | 1 |
| 5 | 1.5 |
| 6 | 2 |
| 7 | 2.5 |
| 8 | 3 |

### Result

The results in FIG. 8 demonstrate that the PEG8000 concentrations influence the cut-off: higher concentrations result in lower cut-off. Because this goes in the opposite direction of the divalent metal cation concentration, it is suitable for fine-tuning the system for a certain cut-off.

### Example 8—Effect of DNA Concentration

The influence on the cut-off value by the amount of input DNA was investigated. Input DNA concentrations of 200, 400, 600, 800, 1000, and 1200 ng were tested.
Bead Suspension: 2% PEG8000; 20 mM Tris pH 7.5; Sera-Mag® Magnetic carboxylate-modified beads (50% w/v)
Divalent cation solution: 300 mM Mg²⁺

### Results

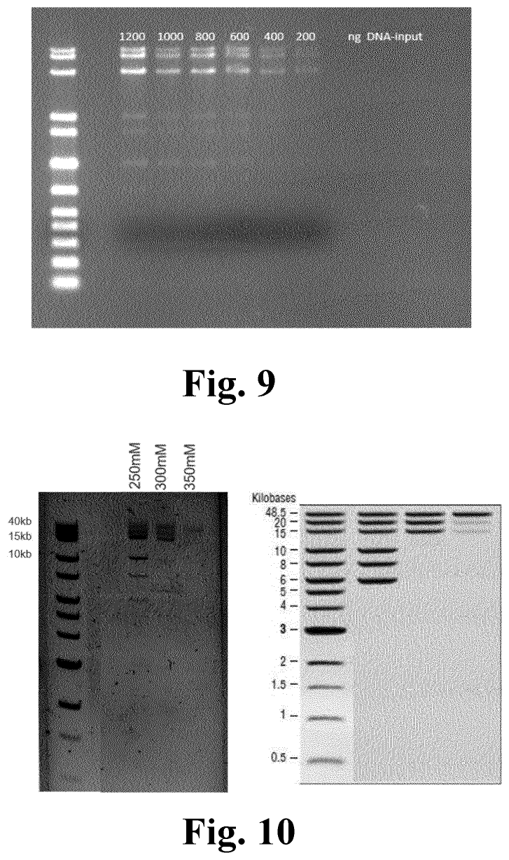

The agarose gel shown in FIG. 9 demonstrates decreasing signals with decreasing amounts of DNA in the size-selective binding over the whole range of added DNA. No change in the cut-off value was observed. This makes the system very versatile.

### Example 9—Range of Scalability

Bead Suspension: 2% PEG8000; 20 mM Tris pH 8; Sera-Mag® SpeedBead Magnetic carboxylate-modified beads (5% w/v)
Divalent cation solution: variable Mg²⁺
Input DNA: Quick-Load® 1kbp Extend DNA Ladder (New England Biolabs)—Size range: 0.5 kbp to 48.5 kbp
Bead suspension/sample ratio: 1:1

### Results

The results of the method of the invention with 250 mM, 300 mM, and 350 mM Mg²⁺ are shown in FIG. 10. The gel itself is shown on the left. However, since the image quality is not the best, a virtual gel with the same results is shown on the right. These data demonstrate the unique property of the system to continuously adjust the cut-off over an extremely wide range of DNA sizes.

### Example 10—Effect of PEG Chain Length

To see if the effect on the cut-off value is dependent on the type of PEG (chain length/molecular weight) or e.g. the number of monomer units present in the binding solution, different PEGs were compared. The different PEGs were used in the standard concentration of 1% (w/v).
Bead Suspension: 2% PEGxxx; 20 mM Tris pH 7.5; Sera-Mag® SpeedBead Magnetic carboxylate-modified beads (5% w/v)
Divalent cation solution: 300 mM Mg²⁺
Input DNA: 1kbp Plus Ladder (QIAGEN)

TABLE 9
Reaction set-ups with PEG with different chain lengths/molecular
weights and different concentrations.

| No. | PEG type | final conc. |
|---|---|---|
| 1 | 8000 | 1 |
| 2 | 6000 | 1 |
| 4 | 4000 | 1 |
| 6 | 3000 | 1 |
| 8 | 2000 | 1 |
| 10 | 1500 | 1 |

### Results

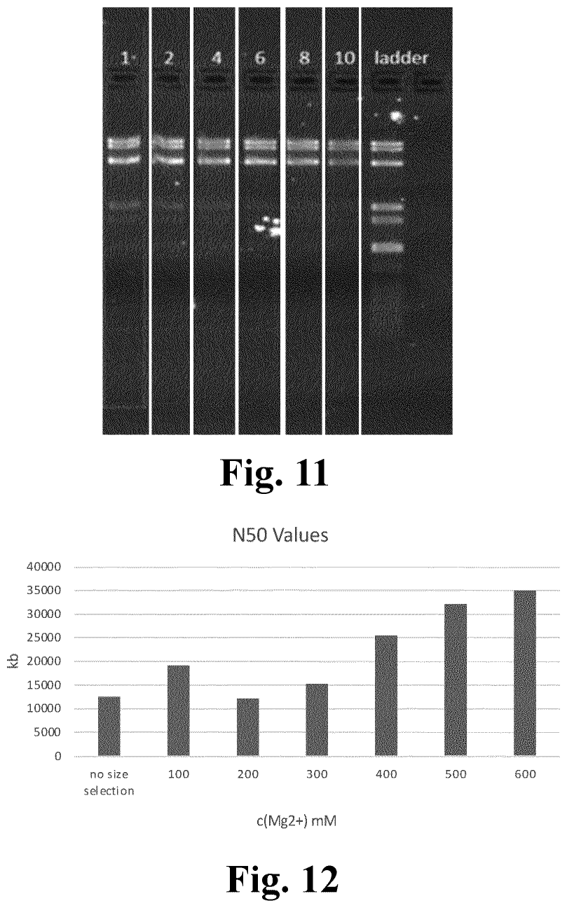

At a concentration of 1% all the different PEGs showed identical size selection behavior (see FIG. 11, lanes 1, 2, 4, 6, 8, and 10).

This demonstrates that the total amount of PEG-units (number of monomers) in the binding solution rather than the number of molecules is the most important factor of the molecular crowding agent with regard to the cut-off value. This indicates a mechanism based on the molecular crowding abilities of PEG which is based on the number of glycol units rather than the number of individual PEG molecules.

### Example 11—Effect of Size Selection on N50 Value in Nanopore Sequencing

An additional way of understanding the value of the removal of smaller fragments is in the improvement of the N50 value in long read sequencing as nanopore sequencing.
Bead Suspension: 2% PEG8000; 20 mM Tris pH 7,5; Sera-Mag® SpeedBead Magnetic carboxylate-modified beads (5% w/V)
Divalent cation solution: variable Mg²⁺
Input DNA: DNA isolated with MagAttract HMW DNA Kit (QIAGEN)
Library Kit: Ligation Sequencing Kit

### Definition of N50 Value:

In ONT sequencing, the N50 value describes the smallest fragment size with which 50% of the total amount of sequenced bases is reached and is therefore a measure of the length of the sequenced fragments: the higher the N50 value, the higher the average length of the sequenced fragments.

The prepared library was sequenced on a MinION Device with a MinION 9.4.1 flowcell.

### Results

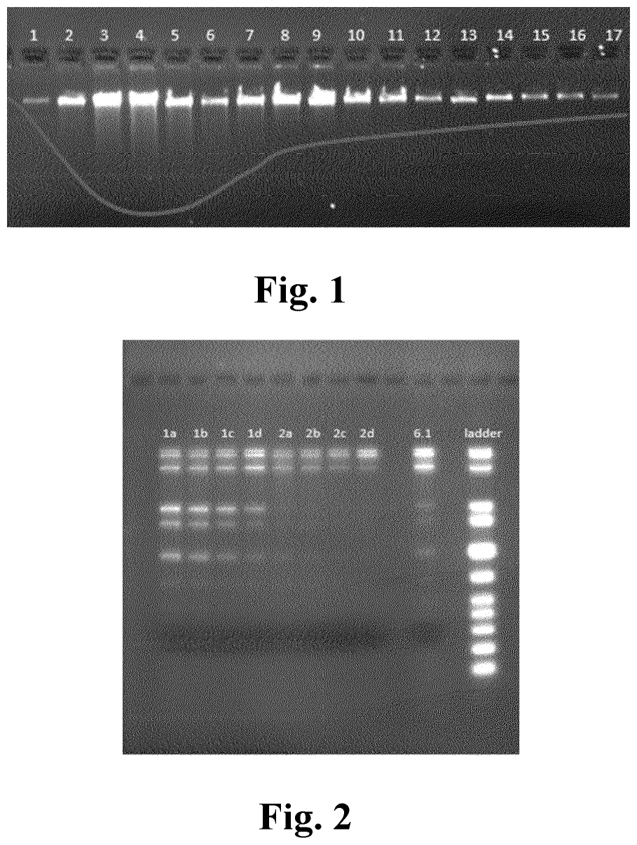

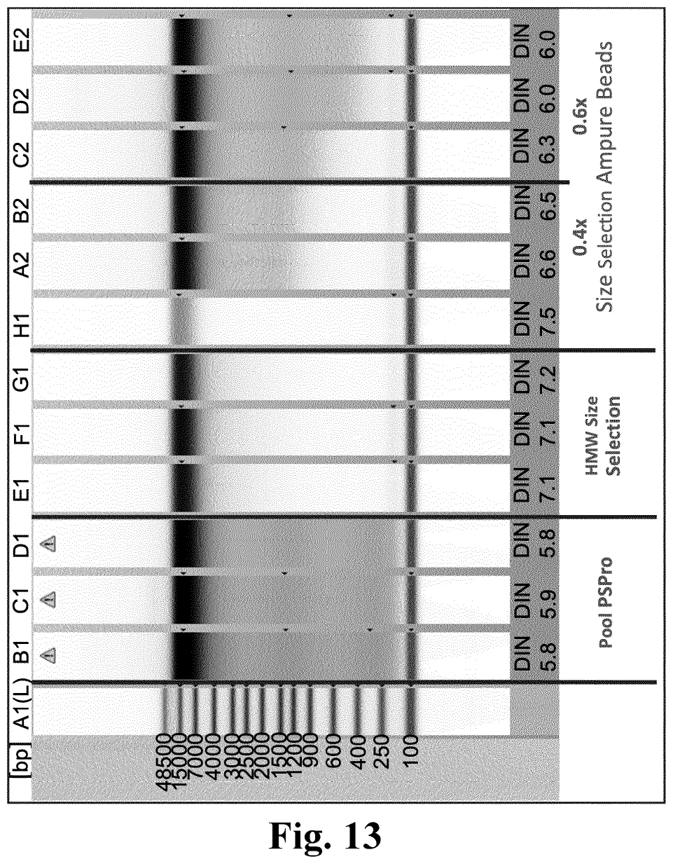

The obtained N50 values are shown in FIG. 12 and demonstrate the effect of the HMW size selection on the N50 value. The decrease from 100 mM to 200 mM followed by increasing values reflects the stationary point (minimum) and transition from the “left side” to the “right side” as shown in FIG. 1, and the curve therein showing the approximate cut-off perfectly reflects the N50 values in FIG. 12. This proves the direct effect of the cut-off on sequencing results.

### Example 12—Effect on Smaller Fragments, Microbiome Analysis

For microbial analysis from environmental samples, relatively harsh lysis conditions like bead beating are necessary to achieve complete lysis even of “difficult-to-lyse” organisms and to get a complete and correct picture of the microbiome. As a consequence, DNA is often sheared resulting in a lot of short sequencing reads and low N50 values, typically <5 kb.

To demonstrate that the size selection of the invention is also beneficial for sequencing of smaller DNA fragments it was tested for microbiome analysis from a soil sample.

For comparison reasons, the well-established AMPure Size Selection (Beckman Coulter Life Science) chemistry (20% PEG, 2.5M NaCl) was used, as well.
Bead Suspension: 2% PEG8000; 20 mM Tris pH 7,5; Sera-Mag® SpeedBead Magnetic carboxylate-modified beads (50% w/v)
Divalent cation solution: 150 mM Mg²⁺
Sample: Soil
DNA Isolation: Power Soil Pro Kit (QIAGEN)
Library Kit: Ligation Sequencing Kit

The prepared library was sequenced on a MinION Device with a MinION 9.4.1 flowcell.

### Results

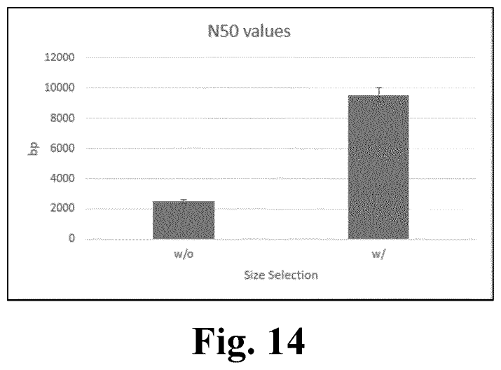

As can be seen in FIG. 13, the size selection system of the invention removes smaller fragments nearly quantitatively. In contrast, the other preparations show a smear down to 100 bp. The samples marked “B1, C1, D1” are without size selection, “E1, F1, G1” are with size selection according to the invention, “0.4×” means that the volume ratio of sample/AMPure is 0.4, and “0.6×” means that the volume ratio of sample/AMPure is 0.6.

FIG. 13 demonstrates that the invention performs significantly better than the well-established AMPure Size Selection. Furthermore, the AMPure Size Selection has parameter values where an increase in volume ratio/AMPure leads to a decrease in cut-off value.

In addition, the obtained N50 values shown in FIG. 14 demonstrate the beneficial effect of the size selection of the invention on the N50 value even for heavily sheared DNA. The N50 value is increased from about 2000 bp to nearly 10 kbp.

1. A method for isolating DNA molecules having a size above a certain cut-off value from a DNA-containing sample, comprising
a) contacting the sample with an aqueous composition comprising beads with a negatively charged surface, a molecular crowding agent, a dissolved salt comprising at least one divalent metal cation, and optionally a buffer for a time sufficient to bind DNA to the surface of the beads;
b) separating the beads with bound DNA from the remaining composition;
c) optionally washing the beads; and
d) optionally eluting the bound DNA from the beads;
wherein the ratio of the concentration of salt to the concentration of beads in the aqueous composition in step a) is at a value where an increase in the ratio leads to an increase in said cut-off value.

2. The method according to claim 1, wherein the negatively charged surface of the beads is a silicate or carboxylate-modified surface.

3. The method according to claim 1, wherein the beads have one or more of the following characteristics:
a) the beads are solid-phase reversible immobilization (SPRI) beads
b) the beads are magnetic beads
c) the beads are SPRI beads with a carboxylate-modified surface

4. The method according to claim 1, wherein the molecular crowding agent is selected from the group consisting of an alkylene glycol, such as ethylene glycol, N-vinylpyrrolidone, oligomers thereof, polymers thereof, and mixtures of said monomers, oligomers, and polymers.

5. The method according to claim 4, wherein the molecular crowding agent has one or more of the following characteristics:
a) it is selected from the group consisting of ethylene glycol, propylene glycol, butylene glycol, pentylene glycol, oligomers thereof, polymers thereof, and mixtures of said monomers, oligomers, and polymers,
b) it is selected from the group consisting of ethylene glycol, diethylene glycol, triethylene glycol, tetraethylene glycol, polyethylene glycol (PEG), and mixtures thereof,
c) it is selected from the group consisting of polyoxyalkylene, such as PEG, polyvinylpyrrolidone and mixtures thereof,
d) it is selected from the group consisting of polyethylene glycol (PEG), polypropylene glycol, polybutylene glycol, polypentylene glycol, and mixtures thereof.

6. The method according to claim 5, wherein the molecular crowding agent is PEG.

7. The method according to claim 6, wherein the PEG has at least one of the following characteristics:
a) the molecular weight of the PEG is in the range of 200 to 35,000 Da, such as in the range of from 1500 to 20,000 Da, e.g. in the range of from 5000 to 12000 Da,
b) the PEG is PEG8000.

8. The method according to claim 1, wherein the concentration of the molecular crowding agent in the aqueous composition is in the range of from 0.1% to 3% (w/v), such as from 0.5% to 2% (w/v), e.g. from 0.75 to 1.25% (w/v).

9. The method according to claim 1, wherein the at least one divalent metal cation has at least one of the following characteristics:
a) it is selected from Mg, Ca, Sr, Co, Ni, Fe and Mn cations, and mixtures thereof,
b) it is selected from Mg, Ca, and Co cations, and mixtures thereof,
c) it is a Mg cation
d) it is selected from Co and Mn cations, and mixtures thereof,
e) it is two or more cations selected from Mg, Ca, Sr, Co, Ni, Fe and Mg cations.

10. The method according to claim 9, wherein the total concentration of the divalent metal cation has at least one of the following characteristics:
(i) It is in the range of from 50 mM to 1000 mM
(ii) It is in the range of from 75 mM to 800 mM
(iii) it is in the range of from 100 mM to 700 mM
(iv) it is in the range of from 120 mM to 600 mM
(v) it is in the range of from 150 mM to 500 mM

11. The method according to claim 1, wherein the ratio of the concentration of salt to the concentration of beads in the aqueous composition is increased by at least 10% compared to the ratio where a small increase neither leads to an increase nor a decrease in cut-off value.

12. The method according to claim 1, wherein the counterion of the divalent cation is selected from the group consisting of halide, such as fluoride, chloride, bromide, or iodide, sulfate, nitrate, carbonate, hydroxide, acetate, and mixtures thereof.

13. The method according to claim 12, wherein the counterion of the divalent cation is selected from the group consisting of fluoride, chloride, bromide, and iodide.

14. The method according to claim 1, wherein the buffer is present in the aqueous composition and provides a pH in the range of from 5-9, such as of from 7 to 9.

15. The method according to claim 1, wherein the DNA bound to the beads has a size between a lower cut-off value and an upper cut-off value

16. The method according to claim 15, wherein the at least one divalent metal cation is selected from Co and Mn, and mixtures thereof.
